(19) United States
(12) Patent Application Publication
(10) Pub. No.: US 2013/0066135 A1
(43) Pub. Date: Mar. 14, 2013

(54) NEUTRON IRRADIATION THERAPY DEVICE

(71) Applicants: Louis Rosa, Tupelo, MS (US); Nicholas Rosa, Tupelo, MS (US); Louis Francis Rosa, Tupelo, MS (US)

(72) Inventors: Louis Rosa, Tupelo, MS (US); Nicholas Rosa, Tupelo, MS (US); Louis Francis Rosa, Tupelo, MS (US)

(21) Appl. No.: 13/598,452

(22) Filed: Aug. 29, 2012

Related U.S. Application Data
(60) Provisional application No. 61/575,825, filed on Aug. 29, 2011.

(51) Int. Cl. A61N 5/10

(52) U.S. Cl. 600/1

(57) ABSTRACT

A device for irradiating cancer patients with neutrons, useful in Boron Neutron Capture Therapy, using at least one neutron emitter mounted and controlled so as to deliver a measured dose of neutrons directed at a treatment site or sites.

### CROSS-REFERENCE TO RELATED APPLICATIONS

This application claims the benefit of U.S. Provisional Application No. 61/575,825, entitled “NEUTRON IRRADIATION THERAPY DEVICE”, filed Aug. 29, 2011, which is hereby incorporated by reference.

### BACKGROUND OF THE INVENTION

1. Field of the Invention

The illustrative embodiments of the invention relate generally to a system for providing neutron radiation to a target treatment site in a clinical environment.

2. Description of Related Art

Generally, external beam radiotherapy for cancer treatment refers to the application of beams of energetic protons, neutrons, or positive ions against a cancerous tumor. Typically, ionizing particles emitted from radiotherapy medical devices are aimed at a target tumor within a patient. The ionizing particles damage the DNA of tumor cells at the target, which causes cell death and thereby reduces tumor size. Cancer cells are uniquely susceptible to this type of therapy because they have a diminished ability to repair damaged DNA.

The current method of radiation therapy typically uses an accelerator, such as a cyclotron, to accelerate ions for use in particle therapy beams. In a cyclotron, charged particles accelerate outward from a center along a spiral path. The particles are held to a spiral trajectory by a static magnetic field and accelerated by a rapidly varying (radio frequency) electric field. This acceleration is provided by an oscillating electric field that is generated between two large, semi-circular plates. A typical cyclotron emits particles along a path that is relatively large in size, and because cyclotrons are typically so large that they occupy entire rooms or buildings, cyclotrons are too large for practical use in most clinical environments for cancer treatment. Further, directing the particles from the cyclotron to a treatment site generally requires establishing a directed ion beam path using, for example, electromagnets.

Neutron Capture Therapy (“NCT”) is a specific type of radiation therapy that focuses on using neutrons to treat cancerous tumors. NCT uses high-energy neutrons to treat various types of cancers and can be advantageous in cancer treatment because NCT causes significant damage to tumors from energetic ions produced by a secondary nuclear reaction after neutrons are absorbed into a nuclide, such as boron isotope ¹⁰B. Other agents may be used in Neutron Capture, such as Gadolinium (Gd). This agent produces gamma rays as well as other products including Auger electrons and offers another pathway for neutron capture. Differences in penetration depths from the resulting products may confer distinct advantages. However there is limited research, due to the availability of neutron sources, making exploration of available agents a slow process.

Most neutron therapy beams are produced from proton beams generated from a cyclotron or other type of particle accelerator directed at a target, such as a beryllium target. Upon being bombarded by the proton beam, the target produces neutrons of different energies, resulting in a neutron beam that can be directed to a tumor site and used for neutron therapy. This method of treatment has been used primarily to treat head and neck tumors.

Boron Neutron Capture Therapy (BNCT) is a specific type of NCT. This approach to treating cancer with neutron radiation involves two steps. The first step in BNCT therapy involves accumulating a boron-containing compound within a tumor. Such accumulation can be accomplished by delivering the boron-containing compound through intravenous, intra-arterial, intraperitoneal, topical, direct tumor injection, and convection-enhanced delivery methods. Specific targeting of the tumor can be accomplished by combining the boron-containing compound to antibodies or receptor-specific ligands that bind to a site on a target protein. Once a predetermined amount of boron-containing compound is established in or on the tumor, the second step of BNCT therapy uses a beam of neutrons that is directed at the boron-containing tumor. The nuclei of ¹⁰B atoms capture the neutrons emitted from the neutron beam. On interacting with the neutrons, the nucleus of the ¹⁰B atom becomes an excited ¹¹B nucleus that rapidly decays to form a high-energy alpha particle and a recoiling lithium ion. The emitted alpha particle causes vast amounts of cellular damage, but only in a range of approximately 10 microns from the decaying boron atom. This high lethality but close proximity to the boron atom, and therefore the cancerous tumor, advantageously results in less damage to neighboring tissues. By delivering ¹⁰B or another neutron capture agent onto cancer cells and subsequently irradiating those cells with a neutron beam, the cancer cells can be preferentially destroyed while minimizing widespread collateral damage to healthy tissue. NCT may also prove promising in targeting tumors located in difficult-to-treat sites (such as across the blood-brain barrier). However, some milestones must be reached before BNCT and NCT can be considered suitable for widespread clinical use.

### SUMMARY

According to an illustrative embodiment, a neutron irradiation device includes a base unit, at least one neutron emitter, and a robotic arm. The robotic arm couples the neutron emitter to the base unit such that the neutron emitter can be positioned to generate and direct a beam of neutrons to a treatment site.

According to another illustrative embodiment, a method for treating a treatment site includes administering a neutron-absorbing material to a treatment site of a patient and directing neutron radiation to the treatment site. The method also includes generating at least one neutron beam using at least one self-contained, low-flux neutron emitter.

In another illustrative embodiment, a system for applying neutron radiation therapy to a treatment site includes at least one self-contained, low-flux neutron emitter and a gantry. Each self-contained, low-flux neutron emitter is coupled to the gantry.

Other features and advantages of the illustrative embodiments will become apparent with reference to the drawings and detailed description that follow.

### BRIEF DESCRIPTION OF THE DRAWINGS

FIG. 1 is a front perspective view of a neutron irradiation device having a neutron emitter mounted to a robotic arm;

FIG. 2 is a front perspective view of a neutron therapy system that includes a neutron irradiation device and a treatment table that supports a patient during the administration of neutron therapy;

FIG. 3 is a side view of a neutron emitter applying a neutron beam to a treatment site inside the body of a patient;

FIG. 4 is an end view of the patient's head;

FIG. 5 is a front perspective view of a neutron therapy system that includes a neutron irradiation device comprising a neutron emitter and a gantry and a treatment table that supports a patient during the administration of neutron therapy;

FIG. 6 is a cross-section view of a neutron emitter;

FIG. 7 is an end view of the system of FIG. 5 delivering a neutron beam to the treatment site that is perpendicular to the surface of a the treatment table;

FIG. 8 is an end view of the neutron beam emitter directing neutron beams to the treatment site from three different angles;

FIG. 9 is a front view of a neutron irradiation device having a plurality of neutron emitters mounted to a robotic arm that, in turn, is mounted to a mobile base unit;

FIG. 10 is a front view of a neutron irradiation device having a plurality of neutron emitters mounted to a gantry that, in turn, is mounted to a mobile base unit;

FIG. 11 is a side view of the neutron irradiation device of FIG. 11, wherein the neutron emitted is rotationally installed within an arc-shaped slot;

FIG. 12 is a side view of the neutron irradiation device of FIG. 11, wherein the neutron emitted is rotationally installed within an arc-shaped slot and rotated to deliver a neutron beam at an oblique angle;

FIG. 13 is a front perspective view of a neutron therapy system that includes a neutron irradiation device comprising a plurality of neutron emitters mounted on a gantry and a moveable treatment table that supports a patient during the administration of neutron therapy;

FIG. 14A is a cross-section view showing the intersection of neutron beams at an initial depth from the neutron emitters;

FIG. 14B is a cross-section view showing the intersection of neutron beams at a second, increased depth from the neutron emitters; and

FIG. 15 is a front perspective view of a neutron therapy system that includes a neutron irradiation device and a treatment table that supports a patient during the administration of neutron therapy, wherein the neutron beam is delivered to the treatment site via a shielded tube.

### DETAILED DESCRIPTION OF THE ILLUSTRATIVE EMBODIMENTS

In the following detailed description of several illustrative embodiments, reference is made to the accompanying drawings that form a part hereof. In several illustrative embodiments, boron and BNCT are discussed are discussed as the operative nuclide and type of Neutron Capture Therapy. In some embodiments, other nuclides, or neutron-capturing molecules, and Neutron Capture Therapies may be substituted as mechanisms for administering neutron therapy. For example, Gadolinium and Gadolinium-based Neutron Capture Therapy (Gd-NCT) may be substituted in many instances.

By way of illustration, the accompanying drawings show specific preferred embodiments in which the invention may be practiced. These embodiments are described in sufficient detail to enable those skilled in the art to practice the invention, and it is understood that other embodiments may be utilized and that logical structural, mechanical, electrical, and chemical changes may be made without departing from the spirit or scope of the invention. To avoid detail not necessary to enable those skilled in the art to practice the embodiments described herein, the description may omit certain information known to those skilled in the art. The following detailed description is, therefore, not to be taken in a limiting sense, and the scope of the illustrative embodiments is defined only by the appended claims.

As described above, BNCT applies concepts from nuclear technology, chemistry, biology, and medicine to deliver targeted cancer treatment to sensitive parts of the human body, such as the head and neck. Advantages of BNCT include the potential ability to selectively deliver an effective radiation dose to a tumor site while exposing the surrounding normal tissues to much lower levels of radiation. This makes BNCT more viable for patients who have already been subjected to other radiation therapies. BNCT can produce striking clinical responses when used to treat patients with particular types of cancers, such as therapeutically refractory head and neck cancers. That said, a number of issues must be resolved before NCT and BNCT can be optimized as a cancer treatment mechanism. First, BNCT would benefit from the development of more tumor-selective boron delivery agents for BNCT, and analogous issues exist with regard to Gd-NCT and other NCTs that require delivery of a specific neutron-absorbing nuclide to a tumor site, but not to surrounding tissue. Second, there is a need for accurate, real time dosimetry to better estimate the radiation doses delivered to the tumor and normal tissues. Third, there is a need for accelerator based neutron sources that can easily be sited in hospitals. Fourth, there is a need for randomized clinical trials. If these four issues can be resolved, NCT will likely have an important place in treating cancers that are loco-regional and that are presently incurable by other therapeutic modalities.

Of the aforementioned issues, two present significant hurdles to establishing effective BNCT treatment processes: (1) delivering sufficient amounts of boron to a given treatment site, and (2) providing a small yet economical source of neutrons for use in a clinical or research setting, capable of supplying neutrons in a controlled manner to the treatment site.

Relating to the first hurdle, one way to deliver sufficient boron to the treatment site is to use boron-containing compounds, such as 4-boronophenylalanine (BPA). In past applications of BNCT, boronated small molecules like BPA were administered in large quantities. However, this treatment method has proven insufficient because boron-containing compounds like BPA deliver an insufficiently small amount of boron to the treatment site. In addition to failing to provide a sufficient amount of boron, large amounts of BPA could lead to the destruction of healthy tissue by the internalized boron delivery agent. To mitigate the destruction of healthy tissue, borane-antibody conjugates that target specific tumors may be administered pre-treatment as monoclonal antibody drugs. Because singly-boronated compounds are unable to deliver sufficient amounts of boron and can destroy healthy tissue, they have been deemed insufficient to effectively treat most tumors.

More recent developments in nano technology have led to advancements in creating compounds with higher concentrations of boron. Boron-nitride nanotubes, for example, are effective delivery agents because each nanotube is approximately 50% boron by molecular composition and about 43.5% boron by weight. Other boron nanostructures, such as those containing boron carbide, may also be effective delivery agents. As a result, boron nanostructures are able to provide a higher boron concentration than boronated molecules like BPA. Because nanostructures may contain, on average, hundreds of thousands of boron atoms, many boron atoms can be delivered to treatment sites utilizing only a single delivery agent. Increasing the number of ¹⁰B atoms delivered to cancer cells increases the efficacy of the therapy while simultaneously decreasing the unwanted effects of the radiation by enabling the application of an effective treatment with a shortened neutron beam exposure time and decreased beam intensity. With greater boron concentrations, and therefore a greater possibility to deliver more boron to treatment sites, the challenge shifts to delivering the high-boron concentration compounds to treatment sites. Binding boron-containing compounds such as boron nanostructures with an appropriate biocompatible conjugate represents one example of a method to deliver boron to a given treatment site. These biocompatible conjugates can allow boron-containing compounds to bind selectively with receptors in cancer cells. Examples of such biocompatible conjugates include combined targeted antibodies or receptor-specific drugs with attached boron or gadolinium-containing materials. Radioisotopes may also be attached to the conjugates to provide targeting confirmation or specific imaging with gadolinium or paramagnetic materials (such as iron) as discussed in more detail below.

Additionally, boron nanostructures attached to cancer cell antibodies may be another effective tool for delivering sufficient boron to the treatment site. The boron nanostructures may employ enriched ¹⁰B isotopes, so as to increase the efficiency of the nanotube-antibody compound in delivering boron to the cancer site. The antibodies bind to the cancer site and facilitate the accumulation of the nanotubes on cancer cells but not elsewhere in the body. Neutrons can then be directed at the cancer site. The neutrons are absorbed by the boron in the nanotubes to form unstable ¹¹B boron nuclei that decay and release a burst of energy to kill the cancer cells. Together, these may exemplify several possible solutions to the first hurdle for acceptance of BNCT for clinical use by facilitating delivery of a sufficient amount of the compounds effective for NCT to treatment sites.

The second hurdle to establishing BNCT therapy as a mainstream treatment process is the lack of an optimized radiation source for supplying a neutron beam that is targeted directly to the treatment site, and utilized in a clinical setting. There exists no practical medical device for use in a clinical setting that emits neutron radiation beams for high volume cancer treatment. Although other treatment methods have proposed the use of a cyclotron, it is, as mentioned before, too impracticably large to use in a clinical setting.

The lack of neutron irradiation sources suitable for providing a narrow beam of neutron radiation to a small treatment site in a clinical setting has been a bar to the wide availability of BNCT therapy. Worldwide, there are only a handful of facilities with the ability to treat humans with neutrons. More problematic is that each of these nuclear facilities relies on isotope neutron sources that require the decay of radioactive material and thus require continuous shielding. This need for shielding and the emission of neutrons of varying energies make the use of nuclear-decay based neutron treatments impractical. The cost and hazard of dealing with radioactive source material has been a further deterrent to construction of additional treatment facilities and thus to the further development of BNCT and other types of NCT. In addition, existing facilities were designed and built for nuclear physics research and thus do not the meet needs of a medical neutron source. One example of such a design mismatch comes in the energies of the neutrons produced. BNCT utilizes epithermal neutrons. While the current facilities do produce epithermal neutrons, they also produce neutrons with thousands of times more energy. The higher energy neutrons produced from these sources must be removed or slowed in order for the neutron beam to be safely harnessed for medical use.

In addition to the need for neutron irradiation sources, the need for a “focused” neutron beam is exemplified by BNCT processes that pair, for example, boron containing nanotubes with trastuzumab (for example, the cancer treatment drug Herceptin), a monoclonal antibody currently used to treat breast cancer. Upon application to a patient, trastuzumab binds to areas other than the treatment site because other bodily tissues also have receptors for the trastuzumab molecule. Cells in the heart, for example, bind to the trastuzumab molecule. This makes it difficult to concentrate the boron-containing compound at a specific and defined treatment site. If a patient is treated with boron-containing compounds conjugated with trastuzumab and unfocused neutron radiation is applied to the entire body, the heart and other tissues in the body that include receptors for the trastuzumab would also sustain tissue damage. To reduce the likelihood of undesired tissue damage, it may be advantageous to apply a two pronged approach of (1) administering materials that bind specifically to the treatment site and (2) applying a “focused” beam of neutron radiation only to the treatment site. Only irradiating specific treatment areas ameliorates concerns about boron-containing compounds binding to undesired sites and being irradiated within the body, thereby causing tissue damage.

It would thus be desirable to have a relatively small, self-contained device that delivers a neutron beam of appropriate energy levels without the expense and size of an isotope neutron source or a linear accelerator. Such a device would be expected to be safer and less expensive to operate. It would allow the development of new facilities for the production of compounds in rapid matter, and for the rapid testing of candidate compounds, without waiting for nuclear reactor time.

Referring now to the Figures, several embodiments of devices that deliver a neutron beam of appropriate energy spectrum without the expense and size of an isotope neutron source or a traditional, non-portable linear accelerator are disclosed. The devices are safer and less expensive to operate than large devices that incorporate such elements. In at least one embodiment, the device delivers neutrons to a patient in a controlled manner so as to provide a desired dose of neutrons for cancer treatment by BNCT at a treatment site. Preferably, the boron-containing compound and its method of delivery are selected so as to limit the presence of the boron containing compound to the cancer cells in and around a tumor to limit the effect of the radiation to the cancer site and reduce extraneous radiation exposure.

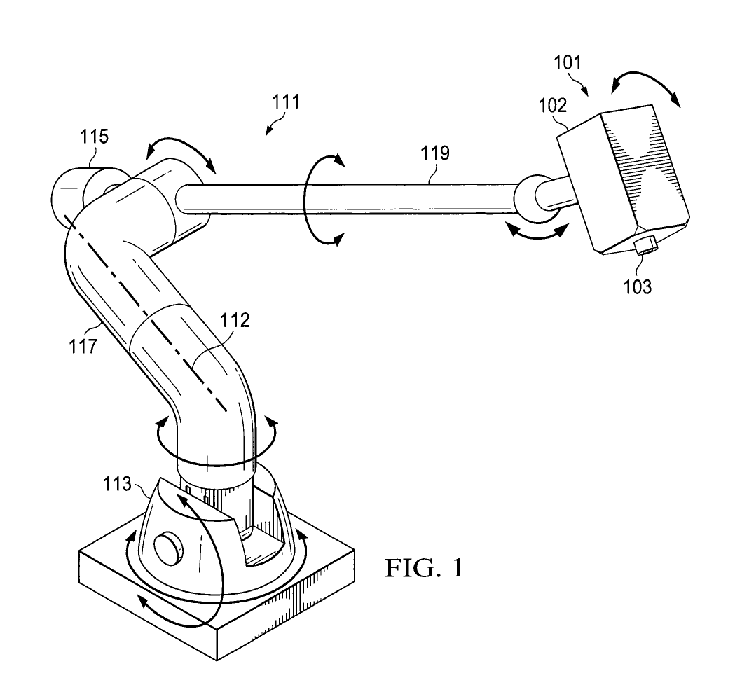

In the embodiments of FIGS. 1-5, a neutron irradiation device 101 comprises at least one low-flux neutron emitter 102 mounted in such a manner as to deliver a controlled dose of neutrons to a specific treatment site in a patient. In some embodiments, the neutron emitters 102 are lower energy range neutron emitters and may produce neutrons from bombardment of beams of deuterium and/or tritium on metal hydride targets sealed in a tube.

In some embodiments, the neutron emitter 102 comprises a portable and compact linear accelerator. In at least one embodiment, the neutron emitter 102 comprises a source to generate positively charged ions, such as a cold-cathode. The neutron emitter 102 has a tubular shape and includes one or more structures to accelerate the ions to a target energy level of, for example, approximately 110 kV. The neutron emitter also includes a metal hydride target loaded with either deuterium, tritium, or a mixture of the two and a gas-control reservoir, also made of a metal hydride material. The cold-cathode is a simple ion source having a hollow cylindrical anode and grounded cathode plates at each end of the anode. In some embodiments, the neutron generator 102 includes a magnet or other element that generates a coaxial electro-magnetic field of several hundred gauss within the cold-cathode. When deuterium and/or tritium gas is introduced proximate the cathode plates at a low pressure of, for example, a few millitorr, the electric field between the anode and cathodes ionizes the gas, creating an ionized plasma. Electrons in the ionized plasma are confined by the electro-magnetic field, which forces the electrons to oscillate back and forth between the cathode plates in helical trajectories while an ion beam is allowed to escape into an acceleration section of the tube through a hole at the center of one of the cathodes. The ion beam may travel through focusing and acceleration elements toward a target, where the ions bombard the target (e.g. a metal hydride) to generate a neutron beam that can be controlled and directed to a treatment site.

In some embodiments, a tubular structure that contains the neutron generating elements is formed by welding, metal brazing, ceramic-to-metal brazing, glass-to-metal sealing, and other joining techniques. The structure may be made from any suitable material, including glass, ceramics, copper, iron, and stainless steel alloys. A benefit of this type of neutron emitter 102 is that it may contain little or no material that emits radiation while the neutron emitter is not operational, and relatively small amounts of radiation as compared to other radiation sources. As a result, this type of neutron emitter may require far less shielding than typical radiation sources, such as a large linear accelerator or a cyclotron.

In one embodiment, a single neutron emitter 102 delivers a flux up to approximately 10⁷ to 10⁹ neutrons per cm² per second. The generally accepted neutron energy ranges for BNCT therapy are approximately 1 eV to 10 keV, as a wide range in energy levels may be desirable to provide an effective dose of neutrons depending on the depth of a tumor. Epithermal neutrons of the 10 keV energy range can be generated by a deuterium-tritium (D-T) source of the type described previously. The neutron emitter 102 may include a neutron generator comprising a linear accelerator that produces neutron flux in the 2.5×10⁹ neutrons/cm²-second range. The neutron emitter 102 alternatively includes a compact neutron generator that produces 10⁹ to 10¹⁰ neutrons/cm²-sec. The compact neutron generator may be combined with a linear accelerator to produce additive flux rates, which may decrease treatment times. For some types of treatments, for example a brain tissue treatment, a flux of 10¹¹ neutrons/cm²-sec may be appropriate based on radiation safety limitations and boron concentrations of 30 ppm for. For other treatments, the flux may be varied depending on the type of tissue being treated as well as different concentrations of boron-containing moieties. As referenced herein, the aforementioned types of neutron emitters may be referred to as “low-flux” neutron emitters.

Examples of devices that may be suitable include neutron generators available from companies such as Adelphi, Thermo Scientific and from NSD Fusion GmbH, including Adelphi DD108, Adelphi DD109, and Thermo D711. In at least one embodiment, the neutron generator produces neutrons with the correct neutron energy values and appropriate output flux rates. However, larger sized neutron generators may not be suitable for certain treatment configurations, such as configurations involving a plurality of generators arranged in an array. Larger neutron generators, however, may be suited for mounting within the neutron emitter 102 as a solitary neutron source. Where larger neutron generators can be accommodated, larger devices having flux rates on the order of 10¹² to 10¹⁴ neutrons/cm²-sec may be used to provide the highest neutron flux levels without requiring a nuclear reactor or a spallation source. Smaller devices, however, may be mounted in an array so that their beams are combined to function as a common neutron source having a higher overall flux. For example, smaller and lighter generators such as Thermo Scientific's Thermo API 120 and Thermo MP320, and NSD Fusion GmbH's NSD-Fusion NSD-350 TT, NSD-350DD, and NSD-350DT may be suited for adaptation into an array. However, these devices have not previously been adapted for medical use and are generally not configured to provide the precise positioning and delivery of neutron radiation needed for cancer therapy.

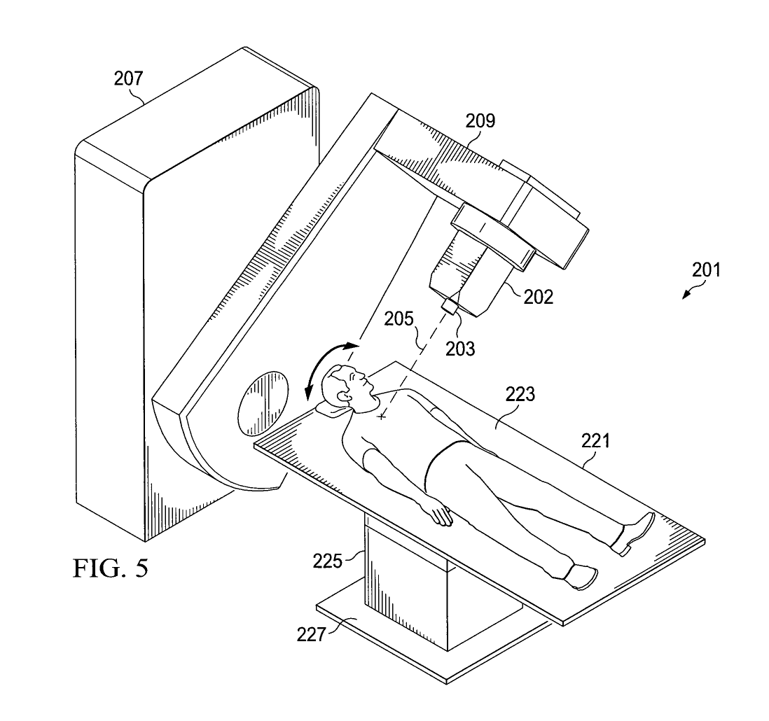

In one embodiment, the neutron generator is a self-contained neutron generator adapted for medical use to provide the ability to treat a specific target with a “focused” beam or beams of neutron radiation. In such an embodiment, the neutron beam produced by the emitter may be focused using collimators, moderators, and software. A benefit associated with using this type of neutron emitter 102 is that it does not require steering or routing of an ion beam produced by a remote source to a, for example, beryllium or tungsten target to produce a neutron beam. Rather, a neutron beam is generated within the self-contained neutron generator unit. In at least one embodiment, the neutron generator is light enough to be mounted on a robot arm or gantry system, as shown in FIGS. 1 and 5, respectively. In some instances, the neutron irradiation device 101 uses multiple neutron emitters 102 to provide treatment. In such an embodiment, each neutron emitter 102 may generate a lower level of neutron flux than the total flux desired for treatment. In some embodiments, each neutron emitter 102 in an array of multiple emitters may be switched on or off during treatment to help “shape” the dose distribution of the neutron beams 105 and change the beam path (although not the target treatment site 133) so as to minimize effects on surrounding tissues. The neutron beam 105 may be delivered from different angles to impinge on the target treatment site that is rich in a boron-containing material.

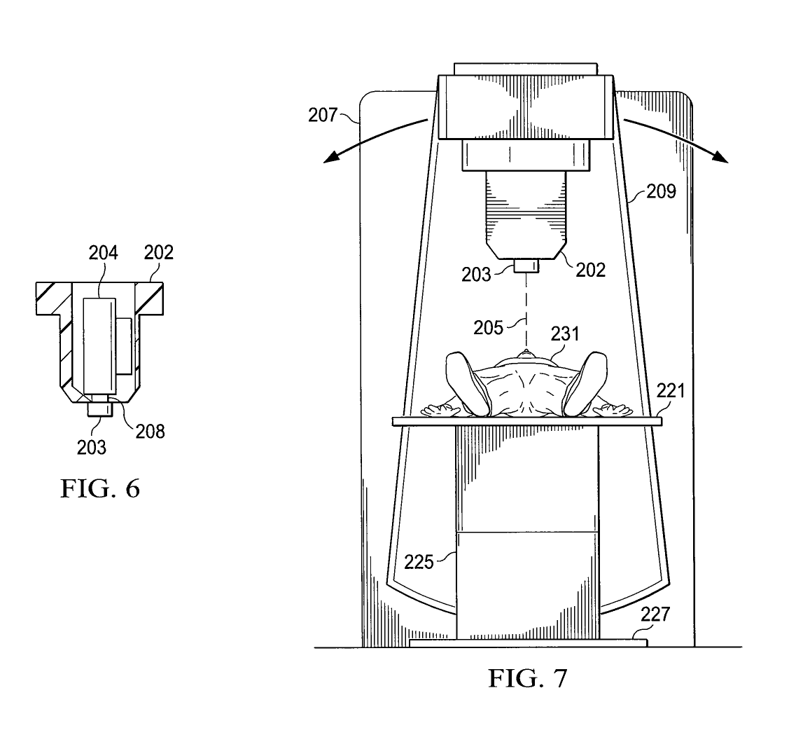

In the embodiment of FIG. 1, the neutron emitter 102 is mounted to a robotic arm assembly 111, and comprises a collimator 103. The low flux neutron emitter 102 may include a moderator similar to the moderator 208 shown in FIG. 6 that incorporates filtering elements, such as layers of lithium, to control the energy level of neutrons. The neutron beam produced the neutron generator passes through the collimator 103 to focus the beam of neutrons supplied by the neutron emitter 102 onto the treatment site. In one embodiment, the neutron emitter 102 is coupled to the robotic arm assembly 111 so that the neutron emitter 102 can be moved and rotated to direct the beam of neutrons. In the embodiment of FIG. 1, the robotic arm assembly 111 comprises a first arm member 117 that is pivotably coupled to a base 113 to allow rotational movement about vertical and horizontal axes through the base. The robotic arm assembly 111 further comprises a second arm member 119 that is rotatably coupled to the first arm member 117 to move about an axis of rotation that is perpendicular to the longitudinal axis 112 of the first arm member 117 and coincident with the intersection of the longitudinal axis of the first arm member and the longitudinal axis of the second arm member. In one embodiment, the robotic arm assembly 111 includes a motor and controller 115 that control the motion and placement of the first robotic arm member 117 and second robotic arm member 119 via a drive or transmission system that is integrated into the arm members 117, 119 and base 113. While the motor and controller 115 shown in FIG. 1 are located at the coupling of the first arm member 117 and the second arm member 119, it is noted that the motor and controller 115 may alternatively be mounted in the base 113 or any other portion of the robotic arm assembly. The neutron emitter 102 is coupled to the second robotic arm member 119 such that the neutron emitter 102 has a 360 degree range of motion relative to the longitudinal axis of the second robotic arm member 119. As such, the neutron emitter 102 may be mounted to the second robotic arm member 119 using any number of couplings, including a ball and socket joint, a bearing mount, a bushing, or a similar coupling. To facilitate electrical connections to the neutron emitter 102, the couplings described herein may comprise a slip ring.

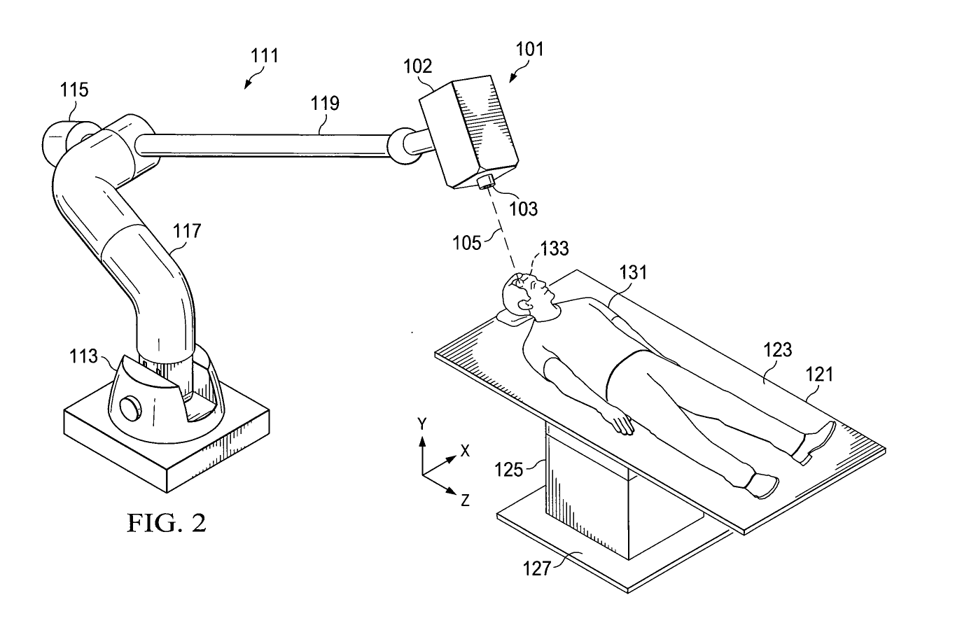

In another embodiment, as shown in FIGS. 2-4, a table 121 is included for the patient 131 to recline upon. The table 121 includes a column 125 that is height adjustable to allow adjustment along the y axis. The column 125 is coupled to a base 127 and a table top 123 that is adjustable along two axes, the x axis and z axis, which are perpendicular to the y axis. The table 121, and thus the patient 131, may be positioned by adjusting the x, y and z axes of movement of the table 121, thereby allowing the treatment site 133 to be brought to an isocenter 137, a point upon which the neutron beam 105 is projected by the neutron irradiation system 101 by virtue of the positioning of the table 121 and neutron emitter 102. In other embodiments, the neutron radiation device 101 comprises neutron emitter(s) 102 mounted on a gantry, robotic arm assembly 111, or other mounting structure that allows for movement over the reclining patient 133. In still other embodiments, the position of one or more of the neutron emitters 102 may be fixed and focused to deliver a treatment dose of the neutron beam 105 to a particular treatment location. In any of these embodiments, targeting of a specific treatment site may be accomplished by moving the table or patient to cause the treatment location to coincide with the treatment site. One advantage of the aforementioned embodiments is that the neutron emitters 102 may be situated to direct a neutron beam directly to a treatment site without the need for a pathway having electromagnetic beam-routing elements, moderators, collimators, or other additional beam processing and shaping elements that are external to the neutron emitter.

The embodiments of the following Figures may include features that are similar to the features of the embodiments discussed above. Such features are generally referred to in the drawings using the same reference numerals as presented in FIGS. 1-4 and indexed by multiples of 100. Referring now to FIGS. 5-8, a neutron irradiation system 201 includes a table 221 to provide a stable platform for a patient 231 that keeps the patient 231 in place to allow for reliable tracking of a target treatment site 233. The table 221 may be rotated about an axis generally parallel to a table surface 223 of the table 221 and passing through the isocenter 237 to change the angle of the table 221 relative to the neutron emitter 202. The table 221 may also slide in longitudinal and transverse directions within the plane of the table surface 223 or be raised and lowered to allow for targeting a different treatment site and easier patient interaction. Information indicating the motion of the table 221 may be tracked by a controller to ensure the correct calibration of the system 201 and delivery of the treatment. For example, the controller may include a memory to track the motion of the table and record the fact that a neutron dose was delivered to the treatment site from a first angle or location before the table was rotated and a second neutron beam was delivered from a second angle or location. In the embodiment of FIGS. 5-8, the neutron emitter 202 is mounted to a gantry 209. The gantry 209, in turn, is pivotably coupled to a base unit 207 such that the gantry 209 and neutron emitter 202 are rotatable about the isocenter 237 of the system 201. The base unit 207 may comprise a counter weight to offset the weight of the gantry 209 and to mechanically stabilize the system 201. While both the gantry 209 and the table 221 have been described as movable to optionally target the treatment site, in at least one embodiment, either the gantry 209 or the table 221 is fixed, thereby allowing targeting of the tissue site by only the movable component.

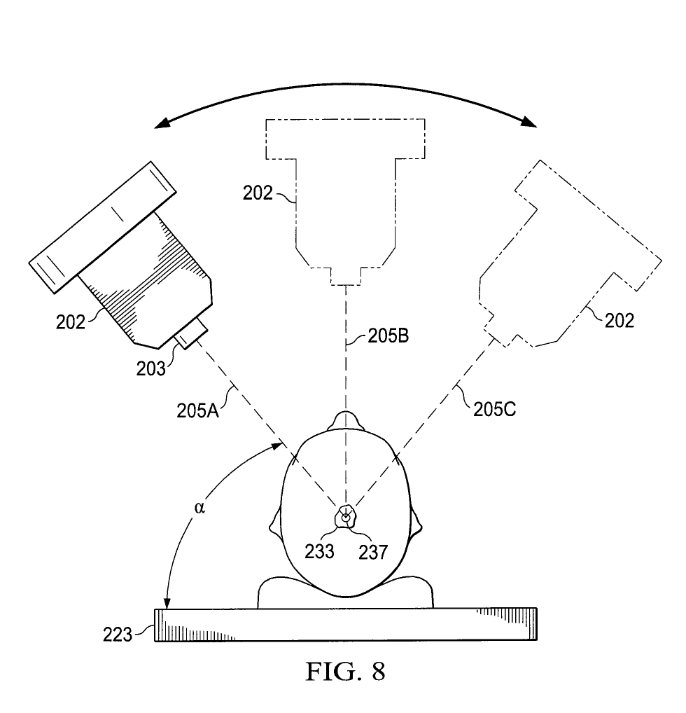

The neutron emitter 202 includes a neutron generator 204 and collimator 203 that direct a beam 205 of neutrons toward the isocenter 237 and coincident portion of the treatment site 233. During operation, rotation of the gantry 209 about the isocenter 237 allows the total irradiation to be spread over a number of individual exposures to a single neutron beam 205 as opposed to a single exposure to multiple neutron beams. As shown in FIG. 8, the rotation results in a distribution of multiple neutron beam paths 205A, 205B, and 205C that are created by using a single neutron emitter 202 directed at the isocenter 237 from different angles, which may also be referred as a focal point that forms at the intersection of the beam paths. Using the configuration of FIG. 8, the single neutron emitter 202 directs multiple neutron beams 205 along multiple beam paths 205A, 205B, 205C to the treatment site 233, thereby delivering a cumulatively stronger dose of neutrons to the target treatment site 233 while subjecting the surrounding tissue to less exposure to the neutron beams 205. One benefit of such an embodiment is the ability to distribute the dose of radiation applied to the patient's skin and other healthy tissue over a wider surface area to limit superficial damage to skin and other healthy tissue while still delivering the desired neutron dose to the target.

Since skin and other healthy tissue will generally be present between the neutron emitter 202 and treatment site 233, the neutron beam characteristics may be varied to consider the dose of neutron radiation applied to this health tissue, as well as the desired depth dose of the neutron beam 205. By varying the angle of neutron beam delivery around the isocenter 237 over the course of treatment, the healthy-tissue dose can be reduced while still providing the desired depth dose at the treatment site 233. Varying the angle of the neutron beam from several separate treatment sources distributes the healthy-tissue dose across a larger surface area.

In at least one embodiment, the neutron generator 204 may produce thermal neutrons having energy values as high as 14 MeV based on D-T fusion reactions. Higher flux devices may also be developed but a combination of neutron generators 204 may be used to increase flux delivery and therefore decrease treatment time without the need for higher energy generators. The energy level and flux rate of the neutron generator 204 may be adapted for use on brain tumors and other potential treatment sites, including superficial targets such as skin lesions, melanoma, and deeper lesions, including but not limited to, lung, liver, pancreas, bowel, and retroperitoneal cancers. The varying densities of these regions will affect the desired energy level and flux rate.

In at least one embodiment, control mechanisms or controllers coordinate the movement of the table 221 and the timing and direction of the neutron beams 205A, 205B, 205C. Such control mechanisms are adapted for use in the neutron irradiation system 201 of, for example, FIG. 8. In one embodiment, the control mechanisms include table controls to adjust the position of the table 221 to locate the target treatment site 233 of the patient 231 at the isocenter 237. The control mechanism may include a patient monitoring system to account for and verify patient motion, such as respiratory motion, and to monitor patient position. In one embodiment, the control mechanism may automatically shut down the neutron irradiation system 201 if patient motion is excessive.

The table controls are communicatively coupled to the table 221 and include motorized elements to adjust the position of the table in the three spatial dimensions x, y, and z, which correspond respectively to the transverse direction of the top table surface 223, the longitudinal direction of the top table surface 223, and the vertical height of the column 225. The table controls may be located on the table 221, such as levers or buttons, or may be accessed via a graphical user interface located on the table 221 or a computer. The control mechanisms also include neutron beam positioning controls to adjust the position of the neutron emitter 202 such that the neutron beam 205 will intersect with the isocenter of the gantry 209 and emitter 202 assembly at an angle α from the plane of the table 221, which may also be referred to as the treatment angle. To adjust the angle α, the gantry 209 or base unit 207 includes a motor or similar device to rotate the gantry 209 about the isocenter 237. The motor is communicatively coupled to the neutron beam positioning controls so that a care giver can adjust the angle α via a user interface. The user interface may include levers, buttons, and a graphical user interface located on the base unit 207 or a computer. Similarly, the system 201 includes neutron beam controls that control the operation of the neutron emitter 202. Using, for example, a graphical user interface, a user specifies the treatment parameters to control the operation of the neutron emitter 202 to activate, deactivate, pulse, and otherwise control the duration of the neutron beam 205. In one embodiment, all of the control mechanisms can be operated through a single user interface.

In at least one embodiment, the table 221 of system 201 includes Velcro straps or other restraints to keep the patient from moving on the table. When the patient is so restrained, the table moves to change the position of the patient as necessary before, during, and after treatment. The table may also be rotatable so that the angle between the table surface and the neutron beam can be adjusted by rotating the table about the isocenter 237. As such, the orientation of the patient 233 on the table should be fixed to allow tracking of the patient 231 by tracking of the table 221. In one embodiment, the system 201 includes movement compensators to change neutron beam 205 activation times and to measure patient movement. The movement compensators are coupled to the control mechanism to keep the target treatment site 233 at the focal point of the neutron beam(s) 205 and compensate for respiratory, cardiac and other small patient movements. In another embodiment, the control system controls movement of the table 221 and the direction and strength of the neutron beam 205 to provide stereotactic treatment to the patient 231. Here, stereotactic treatment refers to precisely directing the neutron beam 205 in three planes using coordinates provided by medical imaging (such as computer tomography) in order to reach a specific locus in the body, for example, the target treatment site 233, as discussed in more detail below.

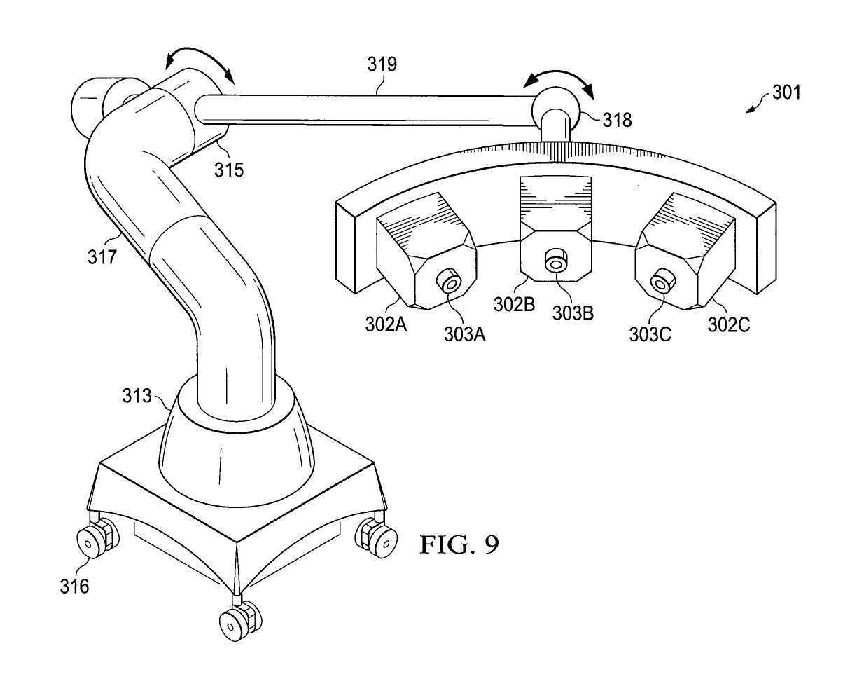

In another illustrative embodiment, as shown in FIG. 9, a neutron irradiation device 301 comprises an arrangement of one or more neutron emitters 302A, 302B, 302C that are mounted to either a stationary or mobile construct, such as a movable base unit 313. The movable base unit 313 may be mounted on wheels 316 for mobility and may be coupled to a movable mounting assembly, such as a robotic arm mount that is similar to the embodiments described previously. The movable base unit 313 may function similarly to the base unit described previously with regard to FIGS. 5-8 insofar as the base unity 313 may include mechanisms to control the positions of the neutron emitters 302A, 302B, 302C and may be coupled to neutron beam positioning controls so that a care giver can adjust the treatment angle via a user interface and specify treatment parameters to control the operation of the neutron emitters 302A, 302B, 302C to activate, deactivate, pulse, and otherwise control the duration of the neutron beams produced by the neutron emitters 302A, 302B, 302C. The robotic arm assembly 315 may include a first arm member 317 mounted to the movable base unit 313 and a second arm member 319 coupled to the first arm member 317. In at least one embodiment, the multiple emitters 302A, 302B, 302C are arranged to provide a focal point of radiation. In another embodiment, the base unit 313 may be configured to support multiple robotic arm assemblies 315. In such embodiments, each robotic arm assembly 315 supports one or more neutron emitters 302 so that each of the neutron emitters 302 can be independently articulated and directed at a treatment site from any angle or distance, irrespective of the angles and distances from which other neutron emitters are directed at the treatment site.

In embodiments having a movable base unit 313, the device 301 is portable (i.e., easier to transport) but may be configured to only generate neutrons when stationary to reduce the risk of unwanted exposure to neutron radiation. As a result, the device 301 may be easier to use and store than other typical radiation systems.

In some embodiments, a plurality of neutron emitters 302 are arranged so as to direct multiple beams 305 at a single locus, or target treatment site thus providing the ability to administer a higher dose of neutrons at a single cancer site, as described previously with regard to FIG. 8. Advantageously, the neutron emitters 302 may be rearranged to deliver a wider beam 305 to a larger area of the patient's body if metastasized cells are being targeted.

Alternatively, the neutron emitters 302 may be mounted in fixed positions such that the neutron beams 305 intersect at a focal point that is coincident with the treatment site, and the patient may be moved to adjust the treatment angle solely by rotating the body of the patient. This embodiment would alleviate any problems associated with moving heavy neutron emitters 302 while maintaining the accuracy of the system 301.

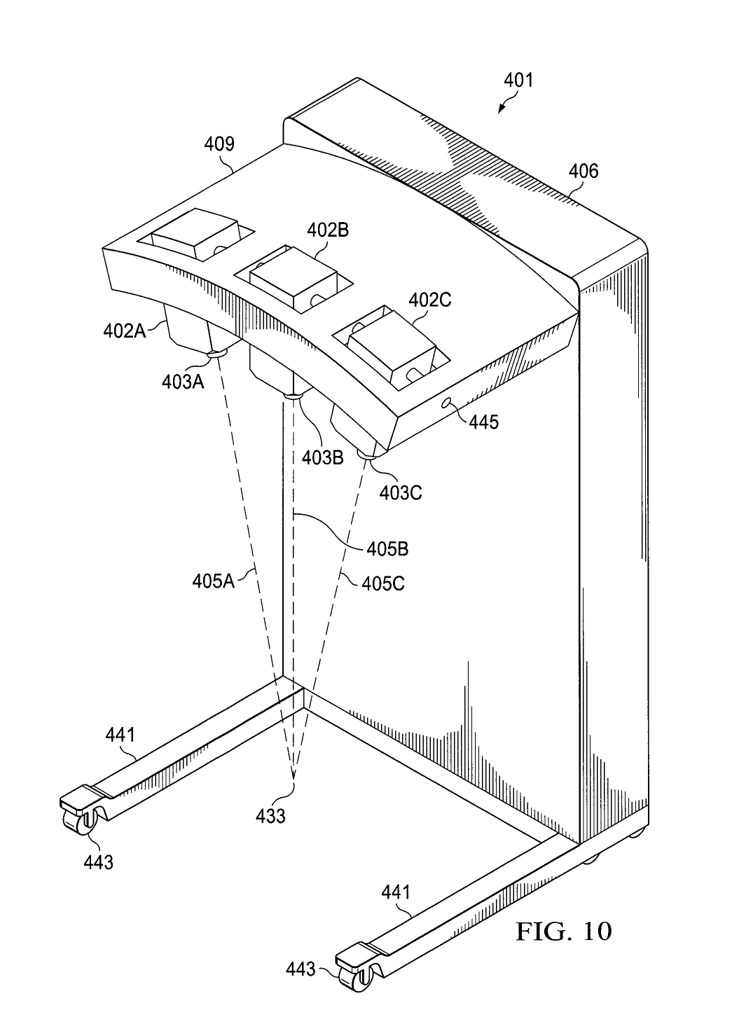

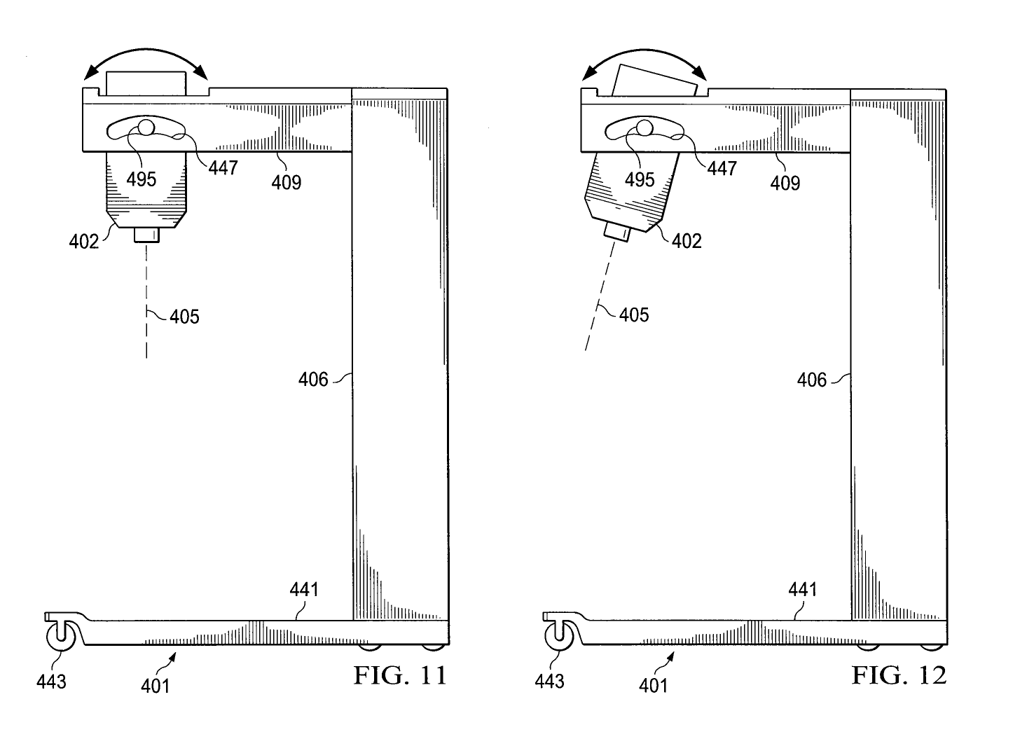

FIGS. 10-12 show a mobile neutron irradiation device 401 comprising a mobile base 406 and gantry 409. To facilitate movement of the focal point 433 of the neutron emitter 402 along an axis that includes the isocenter 437, each neutron transmitter 402 of the array is rotatably mounted to one or more rotational mounts 445 that are included within the gantry 409 of the irradiation device 401. By rotating each of the neutron emitters 402 and moving the mobile base 406 without adjusting the height of the neutron emitters 402, the treatment angle and distance from the focal point 437 to the neutron emitters 402 may change while still addressing the same treatment site, which may allow for varying the amount of radiation received at the treatment site. An advantage to using the irradiation device 401 of FIGS. 10-12 is that the device 401 is able to deliver a therapeutic dose to a specific targeted area at the focal point 433 while minimizing the effects of subjecting other tissues to neutron radiation. These effects can be minimized further by using individual, lower intensity neutron beams 405 that deliver a much lower dose or radiation to the tissue between the surface and the treatment site. In another embodiment, to keep the distance from the neutron emitters 402 to the focal points constant, the rotational mounts 445 may be mounted within arc-shaped slots 447 that provide an additional range of motion. Such slots 447 allow rotational movement about the focal point 433 in a direction that is substantially perpendicular to the array of neutron transmitters, thereby enabling even more possible angles of neutron beam delivery, or treatment angles.

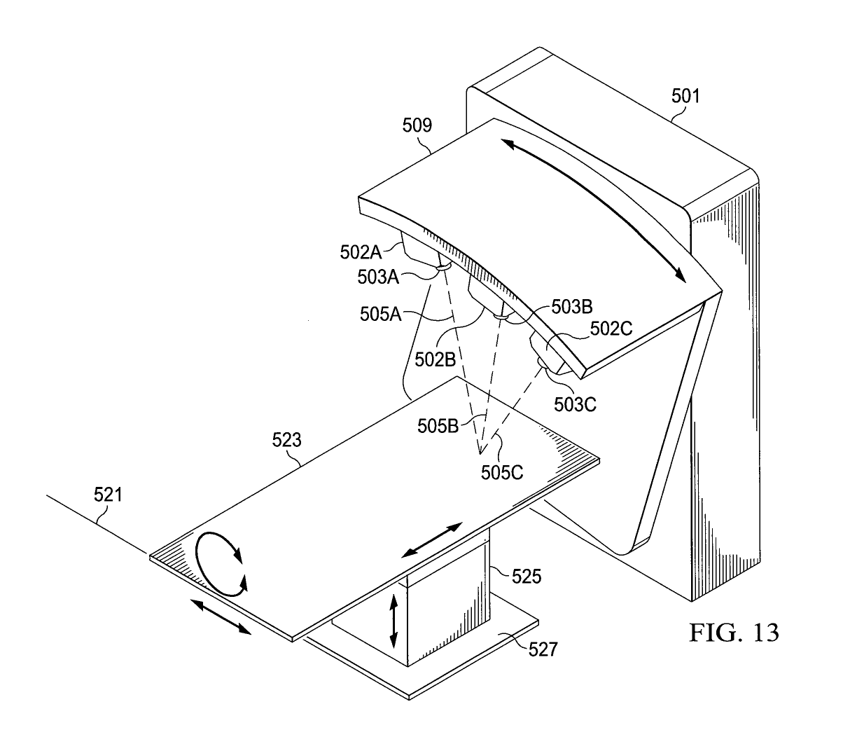

In the embodiment of FIG. 13, a semi-spherical or semi-cylindrical array of multiple, independently-controlled neutron emitters 502A, 502B, 502C is constructed on a stationary neutron irradiation device 501 to give a single focal point of neutron radiation at the intersection of the neutron beams 505A, 505B, 505C produced by the neutron emitters 502A, 502B, 502C, respectively. The targeting of the neutron emitters 502A, 502B, 502C may be manipulated by changing the position of the patient as described previously, by controlling the individual neutron emitters 502A, 502B, 502C, or both. In the embodiment, each component neutron beam path 505A, 505B, 505C would travel a different path through the patient and intersect the other beam paths at the treatment site, which is at the isocenter of the semi-spherical or semi-cylindrical array.

In at least one embodiment, the neutron emitters 502A, 502B, 502C are placed on a gantry 509 or robotic arm that rotates about a single axis, as shown in FIG. 13. This rotation allows the total irradiation to be spread over a number of individual exposures to neutron beams 505, as opposed to a single exposure to more intense neutron beams. The rotation combines the benefit of providing a convenient method of creating the multitude of beam paths 505A, 505B, 505C that are created by an array with the benefit of having adjustable beam paths 505A, 505B, 505C. In yet another embodiment, the emitters 502A, 502B, 502C are placed on a robotic arm that moves and rotates in three dimensions to provide the movement necessary to create a single focal point from multiple beam paths in order to achieve the same effects.

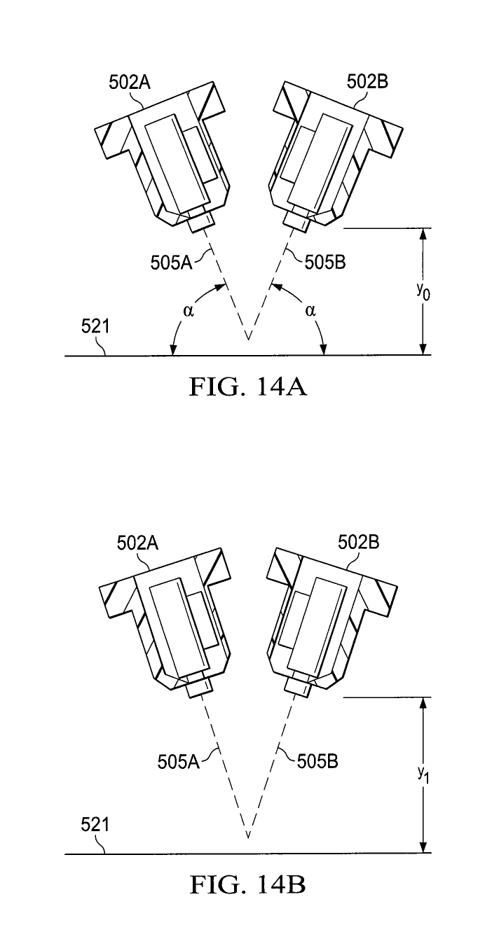

In some embodiments, control mechanisms, or controllers, coordinate the movement of neutron emitters 502A and 502B to adjust an angle α between the neutron emitters 502A, 502B and the plane of the table 521 as shown in FIGS. 14A and 14B. By varying the treatment angle, the depth y of the focal point at which the neutron beam paths 505A and 505B intersect is varied. For example, at an initial angle α, the neutron beam paths 505A, 505B intersect at an initial depth y₀, where the beam paths 505A, 505B form the focal point 533. As the angle α increases, as shown in FIG. 14B, the depth of the focal point 533 increases to an increased depth of y₁. By varying the depth of the focal point 533, different target treatment sites can be treated without varying the height of the table 521.

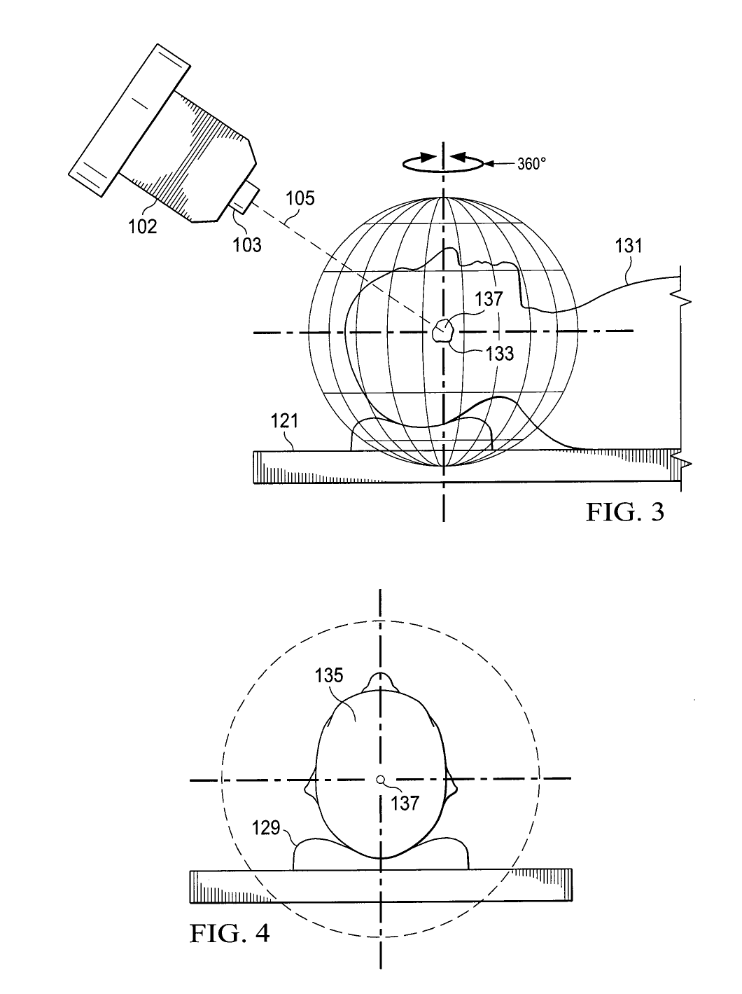

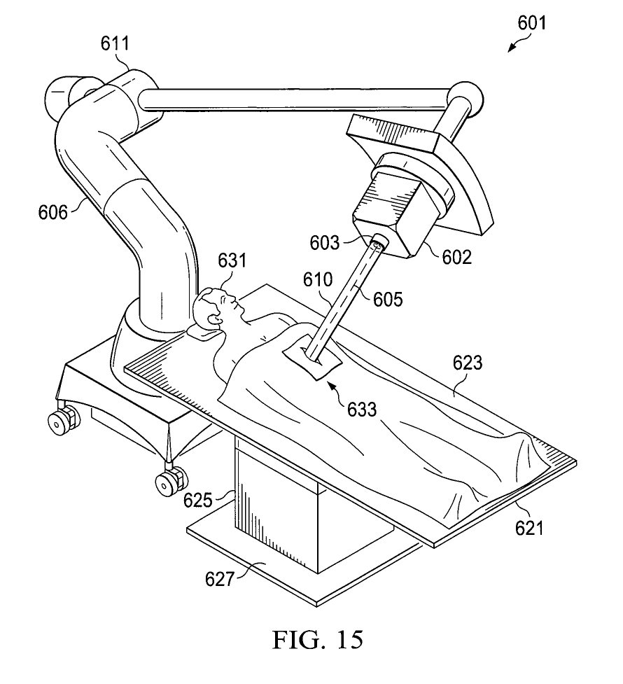

FIG. 15 shows another illustrative embodiment of a system 601 for applying neutron therapy that can be used during an interoperative procedure. In the embodiment, the system 601 may apply neutron radiation therapy during the course of a laparoscopic procedure, such as the removal of a tumor from a colon or gall bladder of a patient 631. During the procedure, a shielding tube 610 is attached to the neutron emitter 602 and inserted into the port used to conduct the surgery. The shielding tube 610 is made from a material that does not transmit neutrons but allows a neutron beam 605 to be transmitted the length of the tube 610, such as a borated polyethylene. In this manner, the neutron therapy may be applied to a specific site without affecting the tissue between the treatment site 633 and the surface of the patient's body 631 because the neutron beam 605 would not travel through body tissue to reach the treatment site 633. The diameter of the tube 610 may be the size of a fist in one embodiment, or much smaller in the case of a smaller incision. The table 621 and neutron emitter 602 are movable by a control system to provide stereotactic neutron therapy, while the tube 610 isolates the patient's bowel or other surrounding tissue to prevent such tissue from being exposed to the neutron beam 605.

Where neutron beams must travel through tissue to reach the treatment site, dose treatment planning will take into account tissue penetration, attenuation through tissue, dose distribution, surface effects, and beam shaping. Doctors familiar with dosing studies will be able to determine the recommended doses for various types and locations of cancer using methods familiar to them. Referring again to FIGS. 3-4, for example, to control the dosage of the neutron radiation received at the treatment site 133, it may be necessary to account for attenuation of the neutron beam 105 as it passes through intermediate tissue between the emitter 102 and the treatment site 133. In one embodiment, filtering elements, such as lithium layers of varying thickness, can be used to form a moderator to obtain a neutron beam 105 that is optimized at a particular energy level. The energy level of the neutron beam 105 dictates the depth to which the beam 105 penetrates the tissue of the patient 131 and the strength of the beam at particular depths. A moderator can be used to optimize the penetration depth of the beam 105 and to ensure that neutrons at the ideal energy level are delivered to the target treatment site. As the beam 105 passes through tissue, the beam 105 may be attenuated up to 50% after a typical depth of, for example, 10-12 cm. This makes varying the angles of treatment important to limit the direct dose to superficial, intermediate structures. Another way to treat deeper tissues is to reduce or even potentially eliminate the superficial doses, by doing the treatments open (in the operating room), as discussed above with regard to FIG. 15. In another embodiment, neutron radiation to the skin or other surrounding tissue may be eliminated by inserting a treatment tube trough a port in the patient's body and delivering the neutron beam 105 through the treatment tube. Such treatment tubes may be economic and disposable and may fit within, over, or around the shielding tube 610 discussed above. In at least one embodiment, the treatment tube and shielding tube 610 would be one device that keeps sensitive tissues, for example the bowel, out of the path of the neutron beam 605. Such an embodiment would be beneficial where a deep tumor requires a high dose of neutron radiation. In such a case, the dose could be delivered by without damaging the skin or other organs that could be in the path of the beam by making an incision to the tumor and bypassing the skin surface while using the shielding tube 605 to isolate and protect organs and tissue from exposure to radiation. In addition, the specific distance to the neutron emitter 102 can be known, and a soft air-filled pillow can be placed at the end of the treatment tube. The treatment tube can be useful for treating intra-abdominal tumors, such as tumors in the in pancreas that may wrap around large arteries, veins and other structures of the abdomen. Unwanted doses can also be reduced by angling the neutron beam source. In supplement to the shielding tube 610, the intervening tissues may also be held in the desired position, out of the path of the treatment beam, by a retractor or other mechanism fitted over the neutron emitter 602 to ensure a clear path from the neutron emitter 602 to the desired target. A benefit to shielding the intervening tissue is that the duration of treatment time may be less critical. The patient may be placed under anesthesia and monitored for respiratory and cardiac-induced movements to ensure that the intervening tissue remains shielded.

Another treatment, intra-peritoneal treatment (direct delivery into the peritoneal cavity) can be done with chemotherapy, but the treatment is painful due to the drug used. Peritoneal metastasis from ovarian cancer is typically fatal, due to the lack of early symptoms and most physicians avoid treatment due to patient discomfort. Using the system of FIG. 15, for example, a receptor-conjugated boron moiety (for example, a boron containing compound that is configured to bind to receptors at the treatment site or other neutron absorbing element, such as gadolinium) can be delivered specifically to the tumor. A dose of activating neutrons, which is designed to be at a high enough energy level to activate the boron but low enough to avoid damaging tissue on its own are delivered to the treatment site from the neutron emitter 602. The activating neutrons should bind to the boron to cause decay and damage to nearby cancer cells, but not cause significant tissue damage to healthy cells located away from treatment site 633. In another embodiment, the receptor conjugated moiety includes a radioisotope or radiopharmaceutical, such as a gallium-67 containing compound, that enables the tumor to be pinpointed using an imaging system such as an MRI system, a gamma camera, a single-photon emission computed tomography system, or similar imaging system. The receptor conjugated moiety thereby serves as a beacon for applying the neutron beam and also attaches the boron or other neutron absorbing element at the tumor. For example, a patient may be given a compound containing a radionuclide prior to surgery to remove a breast tumor. At the time of the surgery, could then use a detector to find the tumor or abnormality and biopsy the area of the abnormality before administering treatment. In an embodiment that includes multiple neutron emitters, such as the embodiment illustrated in FIG. 9, one of the neutron emitters 302B may be replaced by a gamma camera to facilitate the imaging described previously.

In one embodiment, it may be desirable to obtain the maximum amount of boron at the treatment site before applying the neutron beam. To facilitate measurement of the concentration of boron, another molecule can be added to the boron-containing compound, such as gadolinium, and detected and measured using, for example, magnetic resonance imaging.

Gadolinium is a rare-earth metal that possesses paramagnetic and radiological properties. While the gadolinium ion occurring in water-soluble salts is toxic, chelated gadolinium compounds are far less toxic because they exit the body quickly. Because of its paramagnetic properties, solutions of chelated organic gadolinium complexes can be used as intravenously administered gadolinium-based MRI contrast agents (i.e., dye) in medical magnetic resonance imaging. Alternatively, a radioactive nuclide (yttrium or iodine) can be administered with the boron and measured by a gamma camera. Detectors such as gamma cameras can be added to the system 601 to facilitate measurements necessary to optimize the timing of the treatment by determining when the boron concentration is highest at the treatment site 633. In an embodiment, the detection subsystem measures the concentration of the boron compound that is bound at the target treatment site and the concentration of unbound boron compound in the patient's bloodstream. Here, the optimum time to administer the neutron beam is when the difference between the target-bound drug and the non-target bound drug is greatest.

In an embodiment in which the BNCT targeting agent is very effective, the possibility of providing full body radiation for BNCT exists by utilizing, for example, boron nanostructures to which the targeting moieties are attached. Highly precise targeting moieties would include antibodies or proteins which would attach to the cancer cells within the patient. When the targeting moieties are released into the blood stream, boron would accumulate only on the cancer cells where they exist in the body. A neutron beam could then be directed so as to expose the whole body and thereby eliminate metastasized cells.

The neutron emitters described herein are, in at least one embodiment, portable devices that are capable of being moved relative to a patient to target a particular treatment site. The systems that include the emitters may have a single emitter that is fixed or movable relative to a patient. With respect to fixed emitters, the patient may be moved to ensure correct targeting of the treatment site. With respect to movable emitters, the patient or the emitters may be moved to target the treatment site and to change the angle and depth of treatment. In some embodiments, the systems described herein may include multiple emitters that are fixed, gang adjustable/movable, or independently movable. Providing multiple emitters in a single treatment system allows the distribution of the total radiation dose through multiple beams over a larger area, thereby decreasing risk of damage to healthy tissue.

The neutron emitters described herein may also be described as self-contained in that the elements required to generate the neutrons are contained in a central locality unlike the more remote arrangement of components associated with cyclotrons and large linear accelerator systems. For example, use of many large neutron emitters must be implemented with external beam-routing elements that alter the direction or path of the neutron beam produced by the neutron emitter so that it can ultimately be directed toward a treatment site. In at least one embodiment, the “self-contained” neutron emitter includes a beam generator and neutron-generating target spaced apart a distance of approximately no more than five feet, and thereby does not make use of a cyclotron or large linear beam particle accelerator. As such, the self-contained neutron emitter is much smaller than a cyclotron-based neutron generator that is typically a non-portable, permanently-installed neutron generator that occupies multiple rooms or even entire buildings. In another embodiment, the beam generator is spaced apart from the neutron-generating target by a distance of approximately no more than twelve feet.

It should be apparent from the foregoing that an invention having significant advantages has been provided. The invention has been described in an illustrative manner, and it is to be understood that the terminology which has been used is intended to be in the nature of words of description rather than limitation. Obviously, many modifications and variations of the described embodiments are possible in light of the above teachings. It is noted that the concepts described previously with regard to the illustrative embodiments may be combined to enhance their effectiveness. It is, therefore, to be understood that within the scope of the appended claims, the invention may be practiced otherwise than specifically described previously. While the invention is shown in only a few of its forms, it is not so limited and is susceptible to various changes and modifications without departing from the spirit thereof.

What is claimed is:

1. A neutron irradiation device comprising:
a base unit, at least one neutron emitter, and a robotic arm, wherein
the robotic arm couples the neutron emitter to the base unit such that the neutron emitter can be positioned to generate and direct a beam of neutrons to a treatment site.

2. The neutron irradiation device of claim 1, wherein the at least one neutron emitter consists of one neutron emitter and wherein the neutron irradiation device does not include beam-routing elements that are external to the neutron emitter.

3. The neutron irradiation device of claim 1, wherein the at least one neutron emitter comprises three or more neutron emitters.

4. The neutron irradiation device of claim 3, wherein the three or more neutron emitters are arranged in a curve and configured to generate neutron beams that intersect at the treatment site.

5. The neutron irradiation device of claim 1, further comprising one or more additional robotic arms, and wherein the one or more additional robotic arms couples a neutron emitter to the base unit.

6. The neutron irradiation device of claim 1, wherein the at least one neutron emitter is not a cyclotron.

7. A method for treating a treatment site of a patient, the method comprising:
administering a neutron-absorbing material to the treatment site;
generating at least one neutron beam using at least one self-contained, low-flux neutron emitter; and
directing neutron radiation to the treatment site.

8. The method of claim 7, further comprising using a collimator to focus the at least one neutron beam.

9. The method of claim 8, further comprising using a moderator to determine the energy level of the neutron beam.

10. The method of claim 7, wherein generating at least one neutron beam comprises simultaneously generating three or more neutron radiation beams, and wherein the three or more neutron radiation beams intersect at the treatment site.

11. The method of claim 7, further comprising:
placing a shielding tube over the treatment site; and
transmitting a neutron beam through the shielding tube.

12. The method of claim 11, further comprising applying a treatment tube within the shielding tube.

13. The method of claim 7, wherein the neutron-absorbing material comprises boron nanostructures.

14. The method of claim 13, wherein the boron nanostructures are bound by a targeting material.

15. The method of claim 14, wherein the targeting material comprises cancer antibodies.

16. The method of claim 14, wherein the neutron absorbing material further comprises a radioisotope.

17. The method of claim 16, further comprising:
using an imaging device to determine an optimal time for administering the neutron-absorbing material to the treatment site; and
directing neutron radiation to the treatment site at the optimal time.

18. The method of claim 7, wherein directing neutron radiation to the treatment site comprises using a neutron irradiation device having an isocenter, the method further comprising:
positioning the patient such that the treatment site is coincident with the isocenter;
focusing the at least one self-contained, low-flux neutron emitter on the isocenter and directing at least one neutron beam to the treatment site from a first position; and
rotating the at least one self-contained, low-flux neutron emitter about the isocenter and directing at least one second neutron beam to the treatment site from a second position.

19. The method of claim 7, wherein the neutron emitter is not a cyclotron.

20. A system for applying neutron radiation therapy to a treatment site, the system comprising at least one self-contained, low-flux neutron emitter and a gantry, wherein each self-contained, low-flux neutron emitter is coupled to the gantry.

21. The system of claim 20, further comprising a control system, the control system being operable to control each self-contained, low-flux neutron emitter and the gantry.

22. The system of claim 21, wherein the at least one self-contained, low-flux neutron emitter comprises three or more self-contained, low-flux neutron emitters, each of the self-contained, low-flux neutron emitters being coupled to the gantry and operable to rotate about an isocenter of the gantry.

23. The system of claim 22, wherein the self-contained, low-flux neutron emitters are configured to generate neutron beams that intersect at the isocenter of the gantry.

24. The system of claim 20, wherein the gantry is stationary, the system further comprising a treatment table, and wherein:
the treatment table is movable along three axes relative to the gantry, and
the treatment table is rotatable about an isocenter to vary a treatment angle between a surface of the treatment table and a neutron beam generated by the self-contained, low-flux neutron emitter.
